SEQUENCE LISTING (69 sequences)

SEQ ID NO: 1 - DNA, 18 bp, Artificial Sequence
  Synthetic rs429358 (APOE) F3 primer
aggaacaactgaccccgg

SEQ ID NO: 2 - DNA, 13 bp, Artificial Sequence
  Synthetic rs429358 (APOE) B3 primer
ggcgcacccgcag

SEQ ID NO: 3 - DNA, 36 bp, Artificial Sequence
  Synthetic rs429358 (APOE) FIP primer
  misc_feature (1)..(2): Nucleotides not matching with reference genome
  misc_feature (20)..(21): Junction of first and second specific sequences
gaacgtcctccatgtccgcggcacggctgtccaagg

SEQ ID NO: 4 - DNA, 36 bp, Artificial Sequence
  Synthetic rs429358 (APOE) BIP primer
  misc_feature (1)..(2): Nucleotides not matching with reference genome
  misc_feature (20)..(21): Junction of first and second specific sequences
ctcggccgcctggtgcagtagctctggccgagcatg

SEQ ID NO: 5 - DNA, 15 bp, Artificial Sequence
  Synthetic rs429358 (APOE) LoopF primer
gcgccgcctgcagct

SEQ ID NO: 6 - DNA, 14 bp, Artificial Sequence
  Synthetic rs7412 (APOE) F3 primer
cggccagagcaccg

SEQ ID NO: 7 - DNA, 16 bp, Artificial Sequence
  Synthetic rs7412 (APOE) B3 primer
cacgcggccctgttcc

SEQ ID NO: 8 - DNA, 35 bp, Artificial Sequence
  Synthetic rs7412 (APOE) FIP primer
  misc_feature (1)..(2): Nucleotides not matching with reference genome
  misc_feature (20)..(21): Junction of first and second specific sequences
agttctgcaggtcatcggcagcctcgcctcccacc

SEQ ID NO: 9 - DNA, 35 bp, Artificial Sequence
  Synthetic rs7412 (APOE) BIP primer
  misc_feature (1)..(2): Nucleotides not matching with reference genome
  misc_feature (20)..(21): Junction of first and second specific sequences
tccctggcagtgtaccaggcgcgctcgcggatggc

SEQ ID NO: 10 - DNA, 17 bp, Artificial Sequence
  Synthetic rs7412 (APOE) LoopF primer
ccgcttacgcagcttgc

SEQ ID NO: 11 - DNA, 21 bp, Artificial Sequence
  Synthetic rs4343 (ACE) F3 primer
gaaattctctgagctcccctt

SEQ ID NO: 12 - DNA, 22 bp, Artificial Sequence
  Synthetic rs4343 (ACE) B3 primer
tttcgggtaaaactggaggatg

SEQ ID NO: 13 - DNA, 40 bp, Artificial Sequence
  Synthetic rs4343 (ACE) FIP primer
  misc_feature (1)..(2): Nucleotides not matching with reference genome
  misc_feature (20)..(21): Junction of first and second specific sequences
tctggccatcacattcgtcacaagcagaggtgagctaagg

SEQ ID NO: 14 - DNA, 37 bp, Artificial Sequence
  Synthetic rs4343 (ACE) BIP primer
  misc_feature (1)..(2): Nucleotides not matching with reference genome
  misc_feature (20)..(21): Junction of first and second specific sequences
agcccggaaatatgaagaccctctccccgccttgtct

SEQ ID NO: 15 - DNA, 20 bp, Artificial Sequence
  Synthetic rs4343 (ACE) LoopF primer
ggggtttgaatgccttgagc

SEQ ID NO: 16 - DNA, 20 bp, Artificial Sequence
  Synthetic rs6265 (BDNF) F3 primer
tgactactgagcatcaccct

SEQ ID NO: 17 - DNA, 17 bp, Artificial Sequence
  Synthetic rs6265 (BDNF) B3 primer
tgggactctggagagcg

SEQ ID NO: 18 - DNA, 39 bp, Artificial Sequence
  Synthetic rs6265 (BDNF) FIP primer
  misc_feature (1)..(2): Nucleotides not matching with reference genome
  misc_feature (20)..(21): Junction of first and second specific sequences
acgatagaagagctgttggagacgtgtacaagtctgcgt

SEQ ID NO: 19 - DNA, 38 bp, Artificial Sequence
  Synthetic rs6265 (BDNF) BIP primer
  misc_feature (1)..(2): Nucleotides not matching with reference genome
  misc_feature (20)..(21): Junction of first and second specific sequences
tctgttcgaaagtgtcagcctgaatgggcccaaggcag

SEQ ID NO: 20 - DNA, 24 bp, Artificial Sequence
  Synthetic rs6265 (BDNF) LoopF primer
ggcccaatgaagaaaacaataagg

SEQ ID NO: 21 - DNA, 20 bp, Artificial Sequence
  Synthetic rs7349186 (PINK1) F3 primer
ccaacatcatccgggttctc

SEQ ID NO: 22 - DNA, 20 bp, Artificial Sequence
  Synthetic rs7349186 (PINK1) B3 primer
tgggagatgtatcagctcca

SEQ ID NO: 23 - DNA, 38 bp, Artificial Sequence
  Synthetic rs7349186 (PINK1) FIP primer
  misc_feature (1)..(2): Nucleotides not matching with reference genome
  misc_feature (20)..(21): Junction of first and second specific sequences
ccgtggaggcgtgagggcaggccttcacctcttccgtg

SEQ ID NO: 24 - DNA, 36 bp, Artificial Sequence
  Synthetic rs7349186 (PINK1) BIP primer
  misc_feature (1)..(2): Nucleotides not matching with reference genome
  misc_feature (20)..(21): Junction of first and second specific sequences
gagaaggcctgggccatggcccgcgctgctgtcact

SEQ ID NO: 25 - DNA, 19 bp, Artificial Sequence
  Synthetic rs7349186 (PINK1) LoopF primer
tcagggtagtcgaccaggg

SEQ ID NO: 26 - DNA, 16 bp, Artificial Sequence
  Synthetic rs226249 (DJ1) F3 primer
gtgacgcagcgtgagg

SEQ ID NO: 27 - DNA, 16 bp, Artificial Sequence
  Synthetic rs226249 (DJ1) B3 primer
cgctggcgctgacctg

SEQ ID NO: 28 - DNA, 36 bp, Artificial Sequence
  Synthetic rs226249 (DJ1) FIP primer
  misc_feature (1)..(2): Nucleotides not matching with reference genome
  misc_feature (20)..(21): Junction of first and second specific sequences
cagaacgcacgccagcacgccaaggcggcgtgagtc

SEQ ID NO: 29 - DNA, 36 bp, Artificial Sequence
  Synthetic rs226249 (DJ1) BIP primer
  misc_feature (1)..(2): Nucleotides not matching with reference genome
  misc_feature (20)..(21): Junction of first and second specific sequences
gattcagcctggtgtggggttcttcctgcggacgcg

SEQ ID NO: 30 - DNA, 16 bp, Artificial Sequence
  Synthetic rs226249 (DJ1) LoopF primer
cgtccggcctccctca

SEQ ID NO: 31 - DNA, 18 bp, Artificial Sequence
  Synthetic rs2736990 (SNCA) F3 primer
gatgatgagcaggcagtc

SEQ ID NO: 32 - DNA, 23 bp, Artificial Sequence
  Synthetic rs2736990 (SNCA) B3 primer
acagatgacttagtaaatgtctg

SEQ ID NO: 33 - DNA, 45 bp, Artificial Sequence
  Synthetic rs2736990 (SNCA) FIP primer
  misc_feature (1)..(2): Nucleotides not matching with reference genome
  misc_feature (20)..(21): Junction of first and second specific sequences
agttcttcctaaacagctcacaattacatagtcagaaatctgagt

SEQ ID NO: 34 - DNA, 41 bp, Artificial Sequence
  Synthetic rs2736990 (SNCA) BIP primer
  misc_feature (1)..(2): Nucleotides not matching with reference genome
  misc_feature (20)..(21): Junction of first and second specific sequences
tcgtatatgtgtgtaacaggttgcatcagataatggcttac

SEQ ID NO: 35 - DNA, 22 bp, Artificial Sequence
  Synthetic rs34637584 (LRRK2) F3 primer
gccatgattatataccgagacc

SEQ ID NO: 36 - DNA, 24 bp, Artificial Sequence
  Synthetic rs34637584 (LRRK2) B3 primer
cctgaagatagaattatgagacag

SEQ ID NO: 37 - DNA, 38 bp, Artificial Sequence
  Synthetic rs34637584 (LRRK2) FIP primer
  misc_feature (1)..(2): Nucleotides not matching with reference genome
  misc_feature (20)..(21): Junction of first and second specific sequences
tctagtcagcaatctttgcatgaaaccccacaatgtgc

SEQ ID NO: 38 - DNA, 38 bp, Artificial Sequence
  Synthetic rs34637584 (LRRK2) BIP primer
  misc_feature (1)..(2): Nucleotides not matching with reference genome
  misc_feature (20)..(21): Junction of first and second specific sequences
atcattgctcagtactgctgacctgatcacctacctgg

SEQ ID NO: 39 - DNA, 24 bp, Artificial Sequence
  Synthetic rs34637584 (LRRK2) LoopF primer
cattgggatacagtgtgaaaagca

SEQ ID NO: 40 - DNA, 18 bp, Artificial Sequence
  Synthetic rs1871042 (GSTP1) F3 primer
agcaaagcagagcagacc

SEQ ID NO: 41 - DNA, 16 bp, Artificial Sequence
  Synthetic rs1871042 (GSTP1) B3 primer
gcatccaggcagccag

SEQ ID NO: 42 - DNA, 38 bp, Artificial Sequence
  Synthetic rs1871042 (GSTP1) FIP primer
  misc_feature (1)..(2): Nucleotides not matching with reference genome
  misc_feature (20)..(21): Junction of first and second specific sequences
tgcagggactcagccagcggtaggggatgggcttaggc

SEQ ID NO: 43 - DNA, 40 bp, Artificial Sequence
  Synthetic rs1871042 (GSTP1) BIP primer
  misc_feature (1)..(2): Nucleotides not matching with reference genome
  misc_feature (20)..(21): Junction of first and second specific sequences
atcccctgccctgcagatctctcatggatcagcagcaagt

SEQ ID NO: 44 - DNA, 18 bp, Artificial Sequence
  Synthetic rs1871042 (GSTP1) LoopF primer
aggaattgggggcagagg

SEQ ID NO: 45 - DNA, 19 bp, Artificial Sequence
  Synthetic rs1050450 (GXT1) F3 primer
tgttcctccctcgtaggtt

SEQ ID NO: 46 - DNA, 16 bp, Artificial Sequence
  Synthetic rs1050450 (GXT1) B3 primer
tgggccctgacggtgt

SEQ ID NO: 47 - DNA, 40 bp, Artificial Sequence
  Synthetic rs1050450 (GXT1) FIP primer
  misc_feature (1)..(2): Nucleotides not matching with reference genome
  misc_feature (20)..(21): Junction of first and second specific sequences
tgagctgtgcctagggcgccatagatgaaaacccccccga

SEQ ID NO: 48 - DNA, 36 bp, Artificial Sequence
  Synthetic rs1050450 (GXT1) BIP primer
  misc_feature (1)..(2): Nucleotides not matching with reference genome
  misc_feature (20)..(21): Junction of first and second specific sequences
atcccttgagacagcagggcagccgccgcttccaga

SEQ ID NO: 49 - DNA, 18 bp, Artificial Sequence
  Synthetic rs1050450 (GXT1) LoopF primer
gcagttgcagtgctgctg

SEQ ID NO: 50 - DNA, 17 bp, Artificial Sequence
  Synthetic rs4880 (SOD2) F3 primer
tgcgcgttgatgtgagg

SEQ ID NO: 51 - DNA, 18 bp, Artificial Sequence
  Synthetic rs4880 (SOD2) B3 primer
cagcctgcgtagacggtc

SEQ ID NO: 52 - DNA, 36 bp, Artificial Sequence
  Synthetic rs4880 (SOD2) FIP primer
  misc_feature (1)..(2): Nucleotides not matching with reference genome
  misc_feature (20)..(21): Junction of first and second specific sequences
cattggggtatctgggctccttccagggcgccgtag

SEQ ID NO: 53 - DNA, 36 bp, Artificial Sequence
  Synthetic rs4880 (SOD2) BIP primer
  misc_feature (1)..(2): Nucleotides not matching with reference genome
  misc_feature (20)..(21): Junction of first and second specific sequences
gtcggagccagctgcctgctcgcggcgctgactgac

SEQ ID NO: 54 - DNA, 19 bp, Artificial Sequence
  Synthetic rs4880 (SOD2) LoopF primer
aggcagaagcacagcctcc

SEQ ID NO: 55 - DNA, 21 bp, Artificial Sequence
  Synthetic rs1001179 (CAT) F3 primer
cagcagggtctaagtattccg

SEQ ID NO: 56 - DNA, 18 bp, Artificial Sequence
  Synthetic rs1001179 (CAT) B3 primer
ccggagatacccaggagg

SEQ ID NO: 57 - DNA, 36 bp, Artificial Sequence
  Synthetic rs1001179 (CAT) FIP primer
  misc_feature (1)..(2): Nucleotides not matching with reference genome
  misc_feature (20)..(21): Junction of first and second specific sequences
agtagccgaacccagggcggggcaggccaccaacgg

SEQ ID NO: 58 - DNA, 36 bp, Artificial Sequence
  Synthetic rs1001179 (CAT) BIP primer
  misc_feature (1)..(2): Nucleotides not matching with reference genome
  misc_feature (20)..(21): Junction of first and second specific sequences
tacgggcaccccgggccggcggaagggtcccgctct

SEQ ID NO: 59 - DNA, 21 bp, Artificial Sequence
  Synthetic rs1001179 (CAT) LoopF primer
cccggttatcagcatccttca

SEQ ID NO: 60 - DNA, 19 bp, Artificial Sequence
  Synthetic COVID19 N gene F3 primer
tggctactaccgaagagct

SEQ ID NO: 61 - DNA, 20 bp, Artificial Sequence
  Synthetic COVID19 N gene B3 primer
tgcagcattgttagcaggat

SEQ ID NO: 62 - DNA, 41 bp, Artificial Sequence
  Synthetic COVID19 N gene FIP primer
  misc_feature (22)..(23): Junction of first and second specific sequences
tctggcccagttcctaggtagtgacgaattcgtggtggtga

SEQ ID NO: 63 - DNA, 40 bp, Artificial Sequence
  Synthetic COVID19 N gene BIP primer
  misc_feature (22)..(23): Junction of first and second specific sequences
agacggcatcatatgggttgcagcgggtgccaatgtgatc

SEQ ID NO: 64 - DNA, 25 bp, Artificial Sequence
  Synthetic COVID19 N gene LoopF primer
tggactgagatctttcattttaccg

SEQ ID NO: 65 - DNA, 21 bp, Artificial Sequence
  Synthetic COVID19 ORF1ab F3 primer
gaaaagaaaaagcttgatggc

SEQ ID NO: 66 - DNA, 21 bp, Artificial Sequence
  Synthetic COVID19 ORF1ab B3 primer
ggtaagtaaccacaagtagtg

SEQ ID NO: 67 - DNA, 46 bp, Artificial Sequence
  Synthetic COVID19 ORF1ab FIP primer
  misc_feature (24)..(25): Junction of first and second specific sequences
agagttgaaaggcacatttggttgtttatgggtagaattcgatctg

SEQ ID NO: 68 - DNA, 46 bp, Artificial Sequence
  Synthetic COVID19 ORF1ab BIP primer
  misc_feature (23)..(24): Junction of first and second specific sequences
cagacgggcgattttgttaaagccaccttctttagtcaaattctca

SEQ ID NO: 69 - DNA, 20 bp, Artificial Sequence
  Synthetic COVID19 ORF1ab LoopF primer
tggtgacgcaactggataga

(19) United States
(12) Patent Application Publication
(10) Pub. No.: US 2023/0416824 A1
(43) Pub. Date: Dec. 28, 2023

(54) SYSTEMS FOR THE DETECTION OF TARGETED GENE VARIATIONS AND VIRAL GENOMES AND METHODS OF PRODUCING AND USING SAME

(71) Applicant: World Biotech Regenerative Medical Group Limited, Hong Kong SAR (CN)

(72) Inventors: Wing Yin Cornelia MAN, Hong Kong SAR (CN); Wei MA, Hong Kong SAR (CN); Quangui GAO, Hong Kong SAR (CN)

(21) Appl. No.: 18/037,003

(22) PCT Filed: Nov. 17, 2021
(86) PCT No.: PCT/IB2021/060665

§ 371 Date: May 15, 2023

Related U.S. Application Data
(60) Provisional application No. 63/114,844, filed on Nov. 17, 2020.

(51) Int. Cl. C12Q 1/6883; C12Q 1/70

(52) U.S. Cl. CPC C12Q 1/6883; C12Q 1/701; C12Q 2600/156

(57) ABSTRACT

Provided are systems for the detection of targeted gene variations and viral genomes and methods for producing and using the same. Methods include screening of individuals having higher risk of developing dementia (e.g. Alzheimer's disease and Parkinson's disease) and weaker anti-oxidative stress systems who are more vulnerable to coronavirus infection. Also provided are methods for screening for coronavirus genomes for early infection detection without the need of special lab equipment. Isothermal amplification techniques can be used, removing the need of a thermal PCR machine. LAMP technique may be used, significantly changing the pH value of the reaction mix. By including a pH indicator, the successful amplification of target DNA can be visualized, removing the need of detecting DNA products using lab detection systems. Specific primers sets are designed for each target SNP and genome of coronaviruses. This ensures high specificity of the LAMP reaction.

### BACKGROUND OF THE DISCLOSURE

Coronaviruses have become a major public health concern in recent years due to the pandemic outbreak of severe acute respiratory syndrome-related coronavirus (SARS-CoV), Middle East respiratory syndrome—related coronavirus (MERS-CoV) and coronavirus disease 2019 (COVID19) related coronavirus (SARS-CoV-2). There is a huge increase in demand for viral screening systems to identify potentially infected individuals to stop the spread of viruses. The present gold standard for coronavirus detection remains real time PCR and reverse transcriptase PCR (RT-PCR) assays. Several antibody-based lateral flow tests have also been used but generally lack sensitivity compared to PCR-based tests. However, PCR-based assays require the use of laboratory equipment such as PCR machines which limits the scalability of these tests.

The elderly is one of the populations in highest risk of coronavirus infection. Disease severity and mortality are high in aged people infected by coronaviruses. In particular, elderly people with dementia (e.g. Alzheimer's disease and Parkinson's disease) are associated with an even higher risk of getting infected, spreading infection, and having a more serious disease outcome. However, during the time of pandemic, it is recommended that elderly people not go to the hospital and clinics to be tested for dementia. There is a need to screen elderly people with a risk of developing dementia and protect them from coronavirus infection at an early stage.

Furthermore, oxidative stress triggered by virus infection is one of the pathways leading to organ damage and respiratory distress. Therefore, people with weaker systems to handle oxidative stress are also potentially vulnerable to coronavirus infection.

There are studies revealing how different single nucleotide polymorphisms (SNPs) affect the risk of development of dementia and having a weaker anti-oxidative stress system. As a result, screening for the presence of such SNPs in individual's genome may help in screening and identifying those who are more vulnerable to coronavirus infection.

Isothermal amplification techniques are nucleic acid amplification reactions that can be performed at a constant temperature using DNA polymerases with strand displacement activities. Amplification may be completed in a single step by incubating the reaction mixture of primers, substrates, DNA polymerase with strand displacement activity and samples at a constant temperature for 30-60 min Loop-mediated isothermal amplification (LAMP), one isothermal amplification technique, achieves target-specific amplification through the use of 4 to 5 different primers specifically designed to recognize 6 to 8 distinct regions in the target gene region. This increases the specificity of LAMP. LAMP typically amplifies target copies 10⁹-10¹⁰ times in a single reaction. The extensive amplification produces large amount of protons and can affect the pH value of the LAMP reaction mixture significantly. Therefore it may be also possible to detect a successful LAMP reaction simply by using a pH indicator without the need to detect the final DNA product using laboratory equipment.

Altogether, there is an urgent demand to develop point-of-care methods for screening individuals potentially infected by coronaviruses and for screening elderly people with higher risk of having dementia and a weaker anti-oxidative stress system. LAMP technique may be a promising way to be used in domestic kits for identifying such individuals by designing primers specific to the genome of coronaviruses and SNPs associated with increased risk of Alzheimer's, Parkinson's and reduced activity of anti-oxidant genes. Since LAMP does not require PCR machine and its result can be detected by colorimetric changes, it can be performed by untrained individuals and used in point-of-care scenarios.

### SUMMARY

In some embodiments, a nucleic acid screening kit may include: a forward inner primer including a nucleic acid sequence selected from SEQ ID NOS. 3, 8, 13, 18, 23, 28, 33, 37, 42, 47, 52, 57, 62, and 67; and a backward inner primer including a nucleic acid sequence selected from SEQ ID NOS. 4, 9, 14, 19, 24, 29, 34, 38, 43, 48, 53, 58, 63, and 68.

In some embodiments, a nucleic acid screening kit may further include: a forward outer primer including a nucleic acid sequence selected from SEQ ID NOS. 1, 6, 11, 16, 21, 26, 31, 35, 40, 45, 50, 55, 60, and 65; and a backward outer primer including a nucleic acid sequence selected from SEQ ID NOS. 2, 7, 12, 17, 22, 27, 32, 36, 41, 46, 51, 56, 61, and 66.

In some embodiments, a nucleic acid screening kit may further include a Loop-F primer including a nucleic acid sequence selected from SEQ ID NOS. 5, 10, 15, 20, 25, 30, 39, 44, 49, 54, 59, 64, and 69.

In some embodiments, a nucleic acid screening kit may further include a reverse transcriptase. In some embodiments, a nucleic acid screening kit may further include a pH indicator.

In some embodiments, a nucleic acid screening kit may further include: one or more of: a positive control sample; a negative control sample; a DNA polymerase with strand displacing activity; dNTPs; and a reaction buffer.

In some embodiments, a forward inner primer includes the nucleic acid sequence of SEQ ID NO. 3; a backward inner primer includes the nucleic acid sequence of SEQ ID NO. 4; a forward outer primer includes the nucleic acid sequence of SEQ ID NO. 1; and a backward outer primer includes the nucleic acid sequence of SEQ ID NO. 2.

In some embodiments, a nucleic acid screening kit may further include a Loop-F primer including the nucleic acid sequence of SEQ ID NO. 5.

In some embodiments, a method of detecting a target nucleotide sequence in a subject may include: preparing a reaction mixture including a biological sample from the subject; a forward inner primer including a nucleic acid sequence selected from SEQ ID NOS. 3, 8, 13, 18, 23, 28, 33, 37, 42, 47, 52, 57, 62, and 67; a backward inner primer including a nucleic acid sequence selected from SEQ ID NOS. 4, 9, 14, 19, 24, 29, 34, 38, 43, 48, 53, 58, 63, and 68; a DNA polymerase with strand displacing activity; dNTPs; a pH indicator; and a reaction buffer; incubating the reaction mixture to produce an incubated mixture; and measuring one or more of turbidity, fluorescence, and colorimetric change of the incubated mixture to determine a result, wherein: a change in the one or more of turbidity, fluorescence, and colorimetric change indicates a positive result, and a positive result indicates the presence of the target nucleotide sequence.

In some embodiments, a reaction mixture for a method of detecting a target nucleotide sequence in a subject may further include: a forward outer primer including a nucleic acid sequence selected from SEQ ID NOS. 1, 6, 11, 16, 21, 26, 31, 35, 40, 45, 50, 55, 60, and 65; and a backward outer primer including a nucleic acid sequence selected from SEQ ID NOS. 2, 7, 12, 17, 22, 27, 32, 36, 41, 46, 51, 56, 61, and 66.

In some embodiments, a reaction mixture for a method of detecting a target nucleotide sequence in a subject may further include a Loop-F primer including a nucleic acid sequence selected from SEQ ID NOS. 5, 10, 15, 20, 25, 30, 39, 44, 49, 54, 59, 64, and 69.

In some embodiments, a reaction mixture for a method of detecting a target nucleotide sequence in a subject may include a forward inner primer includes the nucleic acid sequence of SEQ ID NO. 3; a backward inner primer includes the nucleic acid sequence of SEQ ID NO. 4; a forward outer primer includes the nucleic acid sequence of SEQ ID NO. 1; a backward outer primer includes the nucleic acid sequence of SEQ ID NO. 2; and a Loop-F primer includes the nucleic acid sequence of SEQ ID NO. 5.

In some embodiments, a nucleic acid screening kit to detect the presence of a target nucleotide sequence in a subject may include a first reaction tube including: a forward inner primer including a nucleic acid sequence selected from SEQ ID NOS. 3, 8, 13, 18, 23, 28, 33, 37, 42, 47, 52, 57, 62, and 67; a backward inner primer including a nucleic acid sequence selected from SEQ ID NOS. 4, 9, 14, 19, 24, 29, 34, 38, 43, 48, 53, 58, 63, and 68; a DNA polymerase with strand displacing activity; dNTPs; and a pH indicator; a positive control tube including: a forward inner primer including a nucleic acid sequence selected from SEQ ID NOS. 3, 8, 13, 18, 23, 28, 33, 37, 42, 47, 52, 57, 62, and 67; a backward inner primer including a nucleic acid sequence selected from SEQ ID NOS. 4, 9, 14, 19, 24, 29, 34, 38, 43, 48, 53, 58, 63, and 68; a DNA polymerase with strand displacing activity; dNTPs; a pH indicator; and a synthetic nucleotide sequence substantially identical to the target nucleotide sequence; and a negative control tube including: a forward inner primer including a nucleic acid sequence selected from SEQ ID NOS. 3, 8, 13, 18, 23, 28, 33, 37, 42, 47, 52, 57, 62, and 67; a backward inner primer including a nucleic acid sequence selected from SEQ ID NOS. 4, 9, 14, 19, 24, 29, 34, 38, 43, 48, 53, 58, 63, and 68; a DNA polymerase with strand displacing activity; dNTPs; and a pH indicator.

In some embodiments, each of the first reaction tube, the positive control tube, and the negative control tube further may include a Loop-F primer including a nucleic acid sequence selected from SEQ ID NOS. 5, 10, 15, 20, 25, 30, 39, 44, 49, 54, 59, 64, and 69.

In some embodiments, a nucleic acid screening kit may further include: a second reaction tube including: a forward inner primer including a nucleic acid sequence selected from SEQ ID NOS. 3, 8, 13, 18, 23, 28, 33, 37, 42, 47, 52, 57, 62, and 67; a backward inner primer including a nucleic acid sequence selected from SEQ ID NOS. 4, 9, 14, 19, 24, 29, 34, 38, 43, 48, 53, 58, 63, and 68; a DNA polymerase with strand displacing activity; dNTPs; and a pH indicator.

In some embodiments, a nucleic acid screening kit to detect the presence of a target nucleotide sequence in a subject may include a first reaction tube including: a forward inner primer including a nucleic acid sequence selected from SEQ ID NOS. 3, 8, 13, 18, 23, 28, 33, 37, 42, 47, 52, 57, 62, and 67; a backward inner primer including a nucleic acid sequence selected from SEQ ID NOS. 4, 9, 14, 19, 24, 29, 34, 38, 43, 48, 53, 58, 63, and 68; a forward outer primer including a nucleic acid sequence selected from SEQ ID NOS. 1, 6, 11, 16, 21, 26, 31, 35, 40, 45, 50, 55, 60, and 65; and a backward outer primer including a nucleic acid sequence selected from SEQ ID NOS. 2, 7, 12, 17, 22, 27, 32, 36, 41, 46, 51, 56, 61, and 66; a DNA polymerase with strand displacing activity; dNTPs; and a pH indicator; a positive control tube including: a forward inner primer including a nucleic acid sequence selected from SEQ ID NOS. 3, 8, 13, 18, 23, 28, 33, 37, 42, 47, 52, 57, 62, and 67; a backward inner primer including a nucleic acid sequence selected from SEQ ID NOS. 4, 9, 14, 19, 24, 29, 34, 38, 43, 48, 53, 58, 63, and 68; a forward outer primer including a nucleic acid sequence selected from SEQ ID NOS. 1, 6, 11, 16, 21, 26, 31, 35, 40, 45, 50, 55, 60, and 65; and a backward outer primer including a nucleic acid sequence selected from SEQ ID NOS. 2, 7, 12, 17, 22, 27, 32, 36, 41, 46, 51, 56, 61, and 66; a DNA polymerase with strand displacing activity; dNTPs; a pH indicator; and a synthetic nucleotide sequence substantially identical to the target nucleotide sequence; and a negative control tube including: a forward inner primer including a nucleic acid sequence selected from SEQ ID NOS. 3, 8, 13, 18, 23, 28, 33, 37, 42, 47, 52, 57, 62, and 67; a backward inner primer including a nucleic acid sequence selected from SEQ ID NOS. 4, 9, 14, 19, 24, 29, 34, 38, 43, 48, 53, 58, 63, and 68; a forward outer primer including a nucleic acid sequence selected from SEQ ID NOS. 1, 6, 11, 16, 21, 26, 31, 35, 40, 45, 50, 55, 60, and 65; and a backward outer primer including a nucleic acid sequence selected from SEQ ID NOS. 2, 7, 12, 17, 22, 27, 32, 36, 41, 46, 51, 56, 61, and 66; a DNA polymerase with strand displacing activity; dNTPs; and a pH indicator.

In some embodiments, a nucleic acid screening kit may further include: a second reaction tube including: a forward inner primer including a nucleic acid sequence selected from SEQ ID NOS. 3, 8, 13, 18, 23, 28, 33, 37, 42, 47, 52, 57, 62, and 67; a backward inner primer including a nucleic acid sequence selected from SEQ ID NOS. 4, 9, 14, 19, 24, 29, 34, 38, 43, 48, 53, 58, 63, and 68; a forward outer primer including a nucleic acid sequence selected from SEQ ID NOS. 1, 6, 11, 16, 21, 26, 31, 40, 45, 50, 55, 60, and 65; and a backward outer primer including a nucleic acid sequence selected from SEQ ID NOS. 2, 7, 12, 17, 22, 27, 32, 36, 41, 46, 51, 56, 61, and 66; a DNA polymerase with strand displacing activity; dNTPs; and a pH indicator.

### DETAILED DESCRIPTION

The present disclosure relates to sets of LAMP primers which may be utilized for nucleic acid screening to detect specific nucleotide sequences. Specifically, LAMP primers of the instant disclosure may be used in nucleic acid screening kits and in methods for detecting one or more target nucleotide sequences from a biological sample. LAMP primers of the instant disclosure may be used in (i) screening nucleic acid samples for SNPs associated with weaker anti-oxidant metabolism and higher risk for neurological diseases and, according to some embodiments, (ii) detecting viral genes from coronaviruses. The present disclosure further relates to methods of using LAMP primer sets. A method may include providing reaction mixtures, which each may include at least one LAMP assay primer set specific for (i) SNPs associated with weaker anti-oxidant metabolism; (ii) SNPs associated with higher risk for neurological diseases; and/or (iii) viral genes from viruses such as coronaviruses. A sample to be analyzed may be mixed with reaction mixtures, incubated under conditions suitable to produce a LAMP assay reaction product, and use visual methods to detect the reaction product.

According to some embodiments, a reaction mixture may include one or more of: at least one LAMP assay primer set; magnesium ion(s); dNTPs; a reaction buffer; a DNA polymerase; a pH indicator; and a saliva sample or nasal swab sample from a subject.

In some embodiments, reverse transcriptase can be included in a reaction mixture to synthesize complementary DNA (cDNA) from viral RNA genomes in the sample for detection.

In some embodiments, a LAMP primer set may include one or more of an F3 primer targeting the ORF lab gene of coronavirus of SEQ ID No 65, a B3 primer of SEQ ID No 66, a FIP primer of SEQ ID No 67, and a BIP primer of SEQ ID No 68. A primer set may include a LoopF primer of SEQ ID No 69.

A sample to be analyzed for presence of the listed SNP and presence of coronavirus nucleic acids may be obtained from a subject. In some embodiments, saliva sample or nasal swab sample may be assayed according to methods of the present disclosure.

The present disclosure includes providing a reaction mixture which may include: a LAMP primer set specific for specific gene variations and genomes of viruses (e.g., coronaviruses), magnesium ion, dNTPs, a reaction buffer, a DNA polymerase and a sample to be analyzed; incubating the reaction mixture under DNA polymerase reactions conditions; specific amplification of reaction product; and detecting the change of pH of the reaction mixture due to successful product amplification by visual color change of a pH indicator. Optionally, a reverse transcriptase can be included in the reaction mixture to perform an RT-LAMP assay. Provided herein are methods for using LAMP assays for detecting the presence of a gene variation and/or viral genome and compositions of LAMP assay kits for detecting the presence of a gene variation and/or viral genomes. Methods and materials of the present disclosure provide a simple, cost-effective and point-of-care alternative to real time PCR/RT-PCR for the rapid screening of individuals with genetic variations and/or may be vulnerable to viral infection and/or are currently infected by coronaviruses. Methods and materials of the present disclosure allow for domestic screening, which may identify individuals in potential risk who may need further medical examinations.

LAMP may be a one-step amplification reaction that amplifies a target DNA sequence with high sensitivity and specificity under isothermal conditions. LAMP has sensitivity similar to traditional real time PCR/RT-PCR and higher efficacy. LAMP reaction mixture includes a DNA polymerase with strand displacement activity and 4 primers targeting 6 specific regions within the target sequence, ensuring specificity of the amplification reaction. A two-nucleotide mismatch can be introduced in the 5′ end of the FIP and BIP primers to detect specific SNP of a target gene. In some embodiments, in order to accelerate LAMP reaction, an additional LoopF primer may be added. In some embodiments, in order to detect presence of RNA viruses, a heat stable reverse transcriptase may be added to the LAMP reaction mixture to perform a reverse transcription loop-mediated isothermal amplification (RT-LAMP).

As the efficacy of LAMP may be high, detection of the amplified product may not be required for identifying a successful LAMP amplification, according to some embodiments. The massive protons produced by the nucleotide incorporation step of LAMP amplification can significantly lower the pH value of the LAMP reaction mixture. Therefore, by addition of a pH indicator to the LAMP reaction mixture, a visual color change of the LAMP reaction mixture can be used to determine if the specific LAMP amplification may be successful or not.

The present disclosure provides a simple and rapid screening system for the detection of targeted gene variations and/or genome of coronaviruses present in saliva sample and/or nasal swab samples from individuals without the need for nucleic acid isolation. Visual color change of the LAMP reaction mixture may be a positive indicator of presence of the targeted gene variations and genome of coronaviruses.

### Primers

Primers directed against targeted gene variants are provided herein which successfully amplify targeted gene variants using standard LAMP and/or accelerated LAMP.

As mentioned above, LAMP reaction mixture may include multiple primers. In some embodiments, a LAMP mixture may contain an even number of primers (e.g., 2 primers, 4 primers, 6 primers, 8 primers, etc.). In some embodiments, a LAMP mixture may contain 4 primers, which may be termed a forward inner primer (FIP), a backward inner primer (BIP), a forward outer primer (F3) and a backward outer primer (B3). In some embodiments, an additional primer, Loop F, may be added to the LAMP reaction mixture to accelerate reaction speed.

Primers for LAMP provided by the present disclosure include nucleic acid sequences which may specifically hybridize to a target gene variant/genome of coronaviruses or the complement of a target gene variant/genome of coronaviruses. For each FIPs and BIPs, two such nucleic acid sequences are joined. In some embodiments a linker may be added at the junction and the linker does not hybridize either to the target gene variant/genome of coronaviruses or the complement of a target gene variant/genome of coronaviruses. The linker can be a nucleic acid or non-nucleic acid moiety which provides flexibility to the junction region. A nucleic acid linker, according to some embodiments, includes 2-6 nucleotides or nucleotide analogs. A non-nucleic acid moiety can be a peptide, carbohydrate, lipid, polyether, polyamide, polyamide, or hydrocarbon.

In some embodiments, a two-nucleotide mismatch may be introduced to the end of FIPs and BIPs comparing to the reference human genome. This allows the specific amplification of target SNP. The first 5′ nucleotide may be matched to the target SNP but not the reference genome. The second 5′ nucleotide may not be matched to either the target SNP or the reference genome to increase specificity of the LAMP reaction. For example if the target SNP has the sequence “5′-CG . . . −3′” and the reference genome has the sequence “5′-CG . . . −3′” (the SNP site may be underlined), the corresponding FIP/BIP primer sequence may be “5′-CA . . . −3′”.

Primers are provided by the present disclosure, which are specific for SNPs associated with risk of Alzheimer's, targeting rs429358 (APOE), rs7412 (APOE), rs4343 (ACE) and rs6265 (BDNF).

Primers are provided by the present disclosure, which are specific for SNPs associated with risk of Parkinson's, targeting rs7349186 (PINK1), rs226249 (DJ1), rs2736990 (SNCA) and rs34637584 (LRRK2).

Primers are provided by the present disclosure, which are specific for SNPs associated with risk of oxidative stress, targeting rs1871042 (GSTP), rs1050450 (GXT1), rs4880 (SOD2) and rs1001179 (CAT).

Primers are provided by the present disclosure, which are specific for genome of coronaviruses, targeting the N gene and ORF1ab.

The present disclosure provides primer sequences having SEQ ID Nos. “1-69”. In some embodiments, a LAMP primer set may be selected from the group consisting of: SEQ ID Nos. 1-69.

A forward inner primer may include a nucleic acid sequence selected from SEQ ID NOS. 3, 8, 13, 18, 23, 28, 33, 37, 42, 47, 52, 57, 62, and 67. A backward inner primer may include a nucleic acid sequence selected from SEQ ID NOS. 4, 9, 14, 19, 24, 29, 34, 38, 43, 48, 53, 58, 63, and 68. A forward outer primer may include a nucleic acid sequence selected from SEQ ID NOS. 1, 6, 11, 16, 21, 26, 31, 35, 40, 45, 50, 55, and 65. A backward outer primer may include a nucleic acid sequence selected from SEQ ID NOS. 2, 7, 12, 17, 22, 27, 32, 36, 41, 46, 51, 56, 61, and 66.

In some embodiments, a LAMP primer set may include one or more of an F3 primer targeting a SNP in APOE (rs429358) of SEQ ID No 1, a B3 primer of SEQ ID No 2, a FIP primer of SEQ ID No 3, and a BIP primer of SEQ ID No 4. A primer set may include a LoopF primer of SEQ ID No 5.

In some embodiments, a LAMP primer set may include one or more of an F3 primer targeting a SNP in APOE (r57412) of SEQ ID No 6, a B3 primer of SEQ ID No 7, a FIP primer of SEQ ID No 8, and a BIP primer of SEQ ID No 9. A primer set may include a LoopF primer of SEQ ID No 10.

In some embodiments, a LAMP primer set may include one or more of an F3 primer targeting a SNP in ACE (rs4343) of SEQ ID No 11, a B3 primer of SEQ ID No 12, a FIP primer of SEQ ID No 13, and a BIP primer of SEQ ID No 14. A primer set may include a LoopF primer of SEQ ID No 15.

In some embodiments, a LAMP primer set may include one or more of an F3 primer targeting a SNP in BDNF (rs6265) of SEQ ID No 16, a B3 primer of SEQ ID No 17, a FIP primer of SEQ ID No 18, and a BIP primer of SEQ ID No 19. A primer set may include a LoopF primer of SEQ ID No 20.

In some embodiments, a LAMP primer set may include one or more of an F3 primer targeting a SNP in PINK1 (rs7349186) of SEQ ID No 21, a B3 primer of SEQ ID No 22, a FIP primer of SEQ ID No 23, and a BIP primer of SEQ ID No 24. A primer set may include a LoopF primer of SEQ ID No 25.

In some embodiments, a LAMP primer set may include one or more of an F3 primer targeting a SNP in DJ1 (rs226249) of SEQ ID No 26, a B3 primer of SEQ ID No 27, a FIP primer of SEQ ID No 28, and a BIP primer of SEQ ID No 29. A primer set may include a LoopF primer of SEQ ID No 30.

In some embodiments, a LAMP primer set may include one or more of an F3 primer targeting a SNP in SNCA (rs2736990) of SEQ ID No 31, a B3 primer of SEQ ID No 32, a FIP primer of SEQ ID No 33, and a BIP primer of SEQ ID No 34.

In some embodiments, a LAMP primer set may include one or more of an F3 primer targeting a SNP in LRRK2 (rs34637584) of SEQ ID No 35, a B3 primer of SEQ ID No 36, a FIP primer of SEQ ID No 37, and a BIP primer of SEQ ID No 38. A primer set may include a LoopF primer of SEQ ID No 39.

In some embodiments, a LAMP primer set may include one or more of an F3 primer targeting a SNP in GSTP1 (rs1871042) of SEQ ID No 40, a B3 primer of SEQ ID No 41, a FIP primer of SEQ ID No 42, and a BIP primer of SEQ ID No 43. A primer set may include a LoopF primer of SEQ ID No 44.

In some embodiments, a LAMP primer set may include one or more of an F3 primer targeting a SNP in GXT1 (rs1050450) of SEQ ID No 45, a B3 primer of SEQ ID No 46, a FIP primer of SEQ ID No 47, and a BIP primer of SEQ ID No 48. A primer set may include a LoopF primer of SEQ ID No 49.

In some embodiments, a LAMP primer set may include one or more of an F3 primer targeting a SNP in SOD2 (rs4880) of SEQ ID No 50, a B3 primer of SEQ ID No 51, a FIP primer of SEQ ID No 52, and a BIP primer of SEQ ID No 53. A primer set may include a LoopF primer of SEQ ID No 54.

In some embodiments, a LAMP primer set may include one or more of an F3 primer targeting a SNP in CAT (rs1001179) of SEQ ID No 55, a B3 primer of SEQ ID No 56, a FIP primer of SEQ ID No 57, and a BIP primer of SEQ ID No 58. A primer set may include a LoopF primer of SEQ ID No 59.

In some embodiments, a LAMP primer set may include one or more of an F3 primer targeting the N gene of coronavirus of SEQ ID No 60, a B3 primer of SEQ ID No 61, a FIP primer of SEQ ID No 62, and a BIP primer of SEQ ID No 63. A primer set may include a LoopF primer of SEQ ID No 64.

In the present disclosure, all primers provided herein which specifically hybridize to a target gene variant/genome of coronaviruses or the complement of a target gene variant/genome of coronaviruses may include at least 13 contiguous nucleotides and, in some embodiments, at least 16 contiguous nucleotides, having a nucleotide sequence at least 95% complementary, or at least 98% complementary, or at least 99% complementary, or at least 100% complementary to 13 or more contiguous nucleotides of a target gene variant/genome of coronaviruses or the complement of a target gene variant/genome of coronaviruses or their complement.

In some embodiments, each FIP and BIP primer provided herein includes two nucleic acid sequences, the first nucleic acid sequence hybridizing to a target SNP/genome of coronaviruses and a second nucleic acid sequence hybridizing to the complement of a target SNP/genome of coronaviruses. In each of these sequences provided herein, the junction of the first and second sequences may be marked by a “-” symbol.

In some embodiments, sets of primers specific for gene variants associated with higher risk of Alzheimer's are provided by the present disclosure for use in standard or accelerated LAMP reactions. In some embodiments, primer sets are detecting gene variants of rs429358 (APOE), rs7412 (APOE), rs4343 (ACE) and rs6265 (BDNF).

A primer set for use in LAMP to detect rs429358 (APOE) may include nucleic acids having SEQ ID Nos. 1-4 or substantially identical nucleic acid sequences. A primer set for use in accelerated LAMP to detect rs429358 (APOE) may include nucleic acids having SEQ ID Nos. 1-5 or substantially identical nucleic acid sequences.

A primer set for use in LAMP to detect rs7412 (APOE) may include nucleic acids having SEQ ID Nos. 6-9 or substantially identical nucleic acid sequences. A primer set for use in accelerated LAMP to detect rs7412 (APOE) may include nucleic acids having SEQ ID Nos. 6-10 or substantially identical nucleic acid sequences.

A primer set for use in LAMP to detect rs4343 (ACE) may include nucleic acids having SEQ ID Nos. 11-14 or substantially identical nucleic acid sequences. A primer set for use in accelerated LAMP to detect rs4343 (ACE) may include nucleic acids having SEQ ID Nos. 11-15 or substantially identical nucleic acid sequences.

A primer set for use in LAMP to detect rs6265 (BDNF) may include nucleic acids having SEQ ID Nos. 16-19 or substantially identical nucleic acid sequences. A primer set for use in accelerated LAMP to detect rs6265 (BDNF) may include nucleic acids having SEQ ID Nos. 16-20 or substantially identical nucleic acid sequences.

Sets of primers specific for gene variants associated with higher risk of Parkinson' s are provided by the present disclosure for use in standard or accelerated LAMP reactions. In some embodiments, primer sets are detecting gene variants of rs7349186 (PINK1), rs226249 (DJ1), rs2736990 (SNCA) and rs34637584 (LRRK2).

A primer set for use in LAMP to detect rs7349186 (PINK1) may include nucleic acids having SEQ ID Nos. 21-24 or substantially identical nucleic acid sequences. A primer set for use in accelerated LAMP to detect rs7349186 (PINK1) may include nucleic acids having SEQ ID Nos. 21-25 or substantially identical nucleic acid sequences.

A primer set for use in LAMP to detect rs226249 (DJ1) may include nucleic acids having SEQ ID Nos. 26-29 or substantially identical nucleic acid sequences. A primer set for use in accelerated LAMP to detect rs226249 (DJ1) may include nucleic acids having SEQ ID Nos. 26-30 or substantially identical nucleic acid sequences.

A primer set for use in LAMP to detect rs2736990 (SNCA) may include nucleic acids having SEQ ID Nos. 31-34 or substantially identical nucleic acid sequences.

A primer set for use in LAMP to detect rs34637584 (LRRK2) may include nucleic acids having SEQ ID Nos. 35-38 or substantially identical nucleic acid sequences. A primer set for use in accelerated LAMP to detect rs34637584 (LRRK2) may include nucleic acids having SEQ ID Nos. 35-39 or substantially identical nucleic acid sequences.

Sets of primers specific for gene variants associated with higher risk of oxidative stress are provided by the present disclosure for use in standard or accelerated LAMP reactions. In some embodiments, primer sets are detecting gene variants of rs1871042 (GSTP), rs1050450 (GXT1), rs4880 (SOD2) and rs1001179 (CAT).

A primer set for use in LAMP to detect rs1871042 (GSTP) may include nucleic acids having SEQ ID Nos. 40-43 or substantially identical nucleic acid sequences. A primer set for use in accelerated LAMP to detect rs1871042 (GSTP) may include nucleic acids having SEQ ID Nos. 40-44 or substantially identical nucleic acid sequences.

A primer set for use in LAMP to detect rs1050450 (GXT1) may include nucleic acids having SEQ ID Nos. 45-48 or substantially identical nucleic acid sequences. A primer set for use in accelerated LAMP to detect rs1050450 (GXT1) may include nucleic acids having SEQ ID Nos. 45-49 or substantially identical nucleic acid sequences.

A primer set for use in LAMP to detect rs4880 (SOD2) may include nucleic acids having SEQ ID Nos. 50-53 or substantially identical nucleic acid sequences. A primer set for use in accelerated LAMP to detect rs4880 (SOD2) may include nucleic acids having SEQ ID Nos. 50-54 or substantially identical nucleic acid sequences.

A primer set for use in LAMP to detect rs1001179 (CAT) may include nucleic acids having SEQ ID Nos. 55-58 or substantially identical nucleic acid sequences. A primer set for use in accelerated LAMP to detect rs1001179 (CAT) may include nucleic acids having SEQ ID Nos. 55-59 or substantially identical nucleic acid sequences.

Sets of primers specific for genome of coronaviruses are provided by the present disclosure for use in standard or accelerated LAMP reactions. In some embodiments, primer sets are detecting the N gene and ORFlab.

A primer set for use in LAMP to detect the N gene may include nucleic acids having SEQ ID Nos. 60-63 or substantially identical nucleic acid sequences. A primer set for use in accelerated LAMP to detect the N gene may include nucleic acids having SEQ ID Nos. 60-64 or substantially identical nucleic acid sequences.

A primer set for use in LAMP to detect ORFlab may include nucleic acids having SEQ ID Nos. 65-68 or substantially identical nucleic acid sequences. A primer set for use in accelerated LAMP to detect ORFlab may include nucleic acids having SEQ ID Nos. 65-69 or substantially identical nucleic acid sequences.

### Biological Sample

The present disclosure can be used to analyze a biological sample from any source (e.g., a subject) for presence of a target gene variant/genome of coronaviruses. A biological sample may be a saliva sample or a nasal swab but can also be blood, plasma, serum, urine and peripheral blood mononuclear cells. In some embodiments, samples can be DNA isolated from biological sample.

In some embodiments of the present disclosure, a sample, such as saliva or nasal swab, may be mixed with a lysis solution to release DNA. The released DNA may be not purified from the sample prior to use in a LAMP reaction provided in the present disclosure. Examples of lysis buffers include alkaline lysis buffer, sodium dodecyl sulfate (SDS) lysis buffer and NP-40 lysis buffer.

### LAMP Reaction

In the present disclosure, LAMP and accelerated LAMP can include one or more sets of primers, dNTPs, a buffer, magnesium ion, a DNA polymerase, a pH indicator and a biological sample to be analyzed for presence of target SNP/genome of coronaviruses. In some embodiments a reverse transcriptase may be added. In some embodiments, a reaction-enhancing additive may be added.

A primer set included in a reaction mixture can be any primer set described above or any primer set that substantially similar to any primer set described above, where substantially similar means having a nucleotide sequence at least 50% identical to, or at least 55% identical to, or at least 60% identical to, or at least 65% identical to, or at least 70% identical to, or at least 75% identical to, or at least 80% identical to, or at least 85% identical to, or at least 90% identical to, or at least 95% identical to, or at least 98% identical to, or at least 99% identical to, or at least 100% identical to any primer set described above. In some embodiments, more than one primer set may be included in a reaction mixture.

Magnesium ion may be added, for example, as magnesium salt such as magnesium chloride or magnesium sulfate.

Any buffer compatible with the reagents and reaction can be used to maintain the LAMP reaction mixture within a pH range suitable for the DNA polymerase to work. Examples of such buffers include phosphate buffers, Tris-HCl/KOH buffers and Tricine buffer.

DNA polymerases which can be added in the reaction mixture are DNA polymerases with strand displacing activity. In some embodiments, DNA polymerases can lack a 5′ to 3′ exonuclease activity. Examples of DNA polymerases used in the present disclosure include Bst DNA polymerase, phi29 DNA polymerase, Klenow DNA polymerase, Vent DNA polymerase, Deep Vent DNA polymerase and their mutants and engineered variants.

Reverse transcriptases which can be added in the reaction mixture include, but are not limited to, reverse transcriptase from Moloney murine leukemia virus (MMLV) and avian myeloblastosis virus (AMV), WarmStart RTx reverse transcriptase, and their mutants and engineered variants.

Reaction enhancing additives (e.g., DMSO, betaine) may be added in a reaction mixture.

In one example of the present disclosure, a LAMP reaction mixture includes 0.2 μM of each of F3 and B3 primers, 1.6 μM of each of FIP and BIP primers, 0.4 μM of LoopF primer, 0.4M betaine, 8 mM MgSO4, 1.4 mM dNTPs, 1×ThermoPol reaction buffer (New England Biolabs, Ipswich, Ma.), 0.32U Bst DNA polymerase (New England Biolabs, Ipswich, Ma.), 7.5U WarmStart RTx reverse transcriptase (New England Biolabs, Ipswich, Ma.), and 10 μl of a biological sample. The LAMP reaction mixture may be topped up to a volume of 25 μl using laboratory grade water. In some embodiments 1×WarmStart® Colorimetric LAMP Master Mix (New England Biolabs, Ipswich, Ma.) may be used to include a pH indicator in the LAMP reaction mixture.

The LAMP reaction mixture may be incubated at a temperature suitable for activity of the DNA polymerase and the reverse transcriptase to produce an incubated mixture. The temperature depends on the particular enzymes used and the nucleotide sequence of the desired target. The reaction mixture may be incubated at the appropriate temperature for a time suitable for production of amplified nucleic acid. The reaction time will depend on the reaction conditions. In general, reaction temperature may be in the range of about 60-65° C. but can be higher or lower depending on each LAMP reaction mixture. In general, reaction time may be in the range of about 30-60 minutes but can be longer or shorter depending on factors including the amount of template nucleic acid in the sample to be analyzed.

### Detection of Successful LAMP Reaction

In the present disclosure, detection of successful LAMP amplification can be achieved by detection of turbidity, fluorescence and/or colorimetric changes. In general, LAMP reaction mixtures with addition of a sample may be compared with positive and/or negative controls.

In some embodiments, a pH indicator may be used to detect protons released during LAMP amplification. The pH indicator used may be a colorimetric or fluorescent dye which changes visual properties such as its emission and/or absorption wavelengths according to the pH of the contacting fluid. Examples of pH indicators include Fluorescein, Pyranine, pHrodo dye (Thermo Fisher Scientific, Waltham, MA) and neutral red. Measuring one or more of turbidity, fluorescence, and/or colorimetric changes of an incubated mixture may produce a result (e.g., positive, negative), where the result may be based on changes in the turbidity, fluorescence, and/or color. For example, a change in the turbidity, fluorescence, and/or color may indicate a positive result (e.g., successful LAMP reaction) and no (or minimal) change in the turbidity, fluorescence, and/or color may indicate a negative results (e.g., unsuccessful LAMP reaction). A positive result may, according to some embodiments, indicate the presence of the target nucleotide sequence (e.g., the sequence targeted by the primers).

### Screening Kits Utilizing LAMP

The present disclosure provides for kits (e.g., screening kits) which may, according to some embodiments, combine amplification of target sequences with detection of results in a single process. For example, a reaction mixture may change color if a specific target sequence may be present in a sample and amplified. A kit (e.g., screening kit) may include any combination of components including reaction tubes, primers, one or more positive controls, one or more negative controls, and one or more pH indicators (e.g., colorimetric indicators). One of ordinary skill in the art readily recognizes that tubes may include any vessel, compartment, etc. that may be capable of housing a chemical reaction described herein.

In a sample embodiment, a kit may include: two reaction tubes, each including a different primer set; a positive control; and a negative control. In some embodiments, at least one component may be freeze-dried such that the component may be shelf stable. In a sample embodiment, each of the reaction tubes, containing primers, may be freeze-dried. A kit of the present disclosure (e.g., screening kit) may contain any one or more (e.g., a combination of) individual primer or primer set disclosed herein.

A positive control may contain any variation of an FIP, BIP, F3, B3, Loop-F primer, DNA polymerase with strand displacing activity, dNTPs, and a pH indicator. In some embodiments, a positive control may contain a synthetic nucleotide sequence substantially identical to the target nucleotide sequence. A negative control may contain any variation of an FIP, BIP, F3, B3, Loop-F primer, DNA polymerase with strand displacing activity, dNTPs, and a pH indicator.

### EXAMPLES

Some specific example embodiments of the disclosure may be illustrated by one or more of the examples provided herein.

### Example 1: Designing Specific Primer Sets for LAMP Reactions

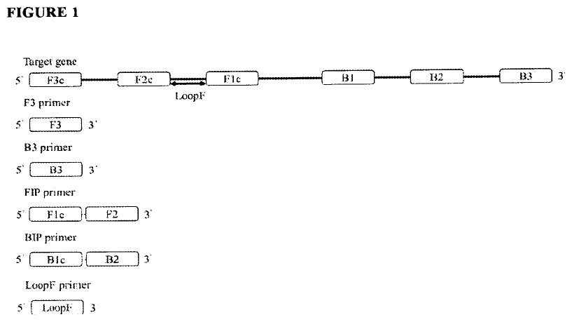

Target gene variant/genome of coronaviruses specific outer primers (F3 and B3), forward inner primer (FIP), backward inner primer (BIP), and loop primer (LoopF) were designed using primer design tools. The sequences of the specific primer sets used in this example are shown below. The symbol “-” in FIP and BIP primers represents the junction of the first and second sequences specifically hybridizing to the target gene variant/genome of coronaviruses. The final primer structures are included in FIG. 1. An example of LAMP primer set targeting rs429358 (APOE) variant may be listed in SEQ ID NOs “1-5”

### Example 2: LAMP Amplification of Target DNA

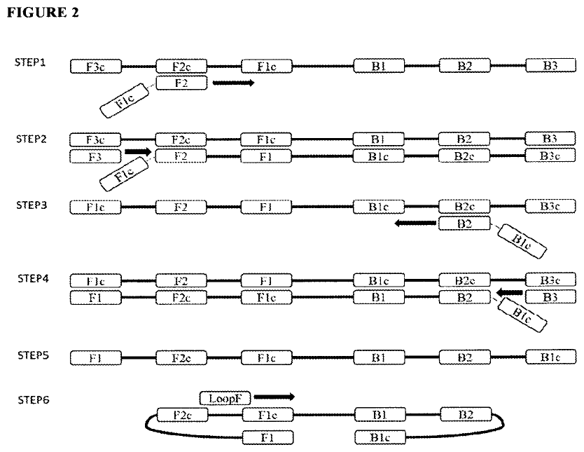

The RT-LAMP reaction to detect rs429358 (APOE) may be carried out in a 25 μl volume, including the following components: 0.2 μM of each of F3 and B3 primers, 1.6 μM of each of FIP and BIP primers, 0.4 μM of LoopF primer, 0.4M betaine, 8 mM MgSO4, 1.4 mM dNTPs, 1×WarmStart® Colorimetric LAMP Master Mix (New England Biolabs, Ipswich, Ma.), 0.32U Bst DNA polymerase (New England Biolabs, Ipswich, Ma.), 7.5U WarmStart RTx reverse transcriptase (New England Biolabs, Ipswich, Ma.), and 10 μl of a biological sample. RT-LAMP amplification may be carried out using a GeneAmp® PCR System (Applied Biosystems, Foster City, Calif.). The reaction mixture may be heated at 60° C. for 60 minutes. Negative controls are included in each run, including a primer only control and/or a water control. Positive control may be also included by adding synthetic SNP DNA. The detailed reaction flow may be illustrated in FIG. 2.

Persons skilled in the art may make various changes without departing from the scope of the instant disclosure. Each disclosed method and method step may be performed in association with any other disclosed method or method step and in any order according to some embodiments. Where the verb “may” appears, it may be intended to convey an optional and/or permissive condition, but its use may be not intended to suggest any lack of operability unless otherwise indicated. Persons skilled in the art may make various changes in methods of preparing and using a composition, device, and/or system of the disclosure. Where desired, some embodiments of the disclosure may be practiced to the exclusion of other embodiments.

Also, where ranges have been provided, the disclosed endpoints may be treated as exact and/or approximations (e.g., read without or with “about”) as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In some embodiments, variation may simply be +/−10% of the specified value. In addition, it may be desirable, in some embodiments, to mix and match range endpoints. Also, in some embodiments, each figure disclosed (e.g., in one or more of the examples, tables, and/or drawings) may form the basis of a range (e.g., depicted value +/− about 10%, depicted value +/− about 50%, depicted value +/− about 100%) and/or a range endpoint. With respect to the former, a value of 50 depicted in an example, table, and/or drawing may form the basis of a range of, for example, about 45 to about 55, about 25 to about 100, and/or about 0 to about 100.

These equivalents and alternatives along with obvious changes and modifications are intended to be included within the scope of the present disclosure. Accordingly, the foregoing disclosure may be intended to be illustrative, but not limiting, of the scope of the disclosure as illustrated by the appended claims. The title, abstract, background, and headings are provided in compliance with regulations and/or for the convenience of the reader. They include no admissions as to the scope and content of prior art and no limitations applicable to all disclosed embodiments.

1. A nucleic acid screening kit comprising:
a forward inner primer comprising a nucleic acid sequence selected from SEQ ID NOS. 3, 8, 13, 18, 23, 28, 33, 37, 42, 47, 52, 57, 62, and 67; and
a backward inner primer comprising a nucleic acid sequence selected from SEQ ID NOS. 4, 9, 14, 19, 24, 29, 34, 38, 43, 48, 53, 58, 63, and 68.

2. The nucleic acid screening kit of claim 1, further comprising:
a forward outer primer comprising a nucleic acid sequence selected from SEQ ID NOS. 1, 6, 11, 16, 21, 26, 31, 35, 40, 45, 50, 55, 60, and 65; and
a backward outer primer comprising a nucleic acid sequence selected from SEQ ID NOS. 2, 7, 12, 17, 22, 27, 32, 36, 41, 46, 51, 56, 61, and 66.

3. The nucleic acid screening kit of claim 1, further comprising a Loop-F primer comprising a nucleic acid sequence selected from SEQ ID NOS. 5, 10, 15, 20, 25, 30, 39, 44, 49, 54, 59, 64, and 69.

4. The nucleic acid screening kit of claim 2, further comprising a Loop-F primer comprising a nucleic acid sequence selected from SEQ ID NOS. 5, 10, 15, 20, 25, 30, 39, 44, 49, 54, 59, 64, and 69.

5. The nucleic acid screening kit of claim 2, further comprising a reverse transcriptase.

6. The nucleic acid screening kit of claim 2, further comprising one or more of:
a positive control sample;
a negative control sample;
a DNA polymerase with strand displacing activity;
dNTPs; and
a reaction buffer.

7. The nucleic acid screening kit of claim 6, further comprising a reverse transcriptase.

8. The nucleic acid screening kit of claim 6, further comprising a pH indicator.

9. The nucleic acid screening kit of claim 2, wherein:
the forward inner primer comprises the nucleic acid sequence of SEQ ID NO. 3;
the backward inner primer comprises the nucleic acid sequence of SEQ ID NO. 4;
the forward outer primer comprises the nucleic acid sequence of SEQ ID NO. 1; and
the backward outer primer comprises the nucleic acid sequence of SEQ ID NO. 2.

10. The nucleic acid screening kit of claim 9, further comprising a Loop-F primer comprising the nucleic acid sequence of SEQ ID NO. 5.

11. A method of detecting a target nucleotide sequence in a subject, the method comprising:
preparing a reaction mixture comprising:
a biological sample from the subject;
a forward inner primer comprising a nucleic acid sequence selected from SEQ ID NOS. 3, 8, 13, 18, 23, 28, 33, 37, 42, 47, 52, 57, 62, and 67;
a backward inner primer comprising a nucleic acid sequence selected from SEQ ID NOS. 4, 9, 14, 19, 24, 29, 34, 38, 43, 48, 53, 58, 63, and 68;
a DNA polymerase with strand displacing activity;
dNTPs;
a pH indicator; and
a reaction buffer;
incubating the reaction mixture to produce an incubated mixture; and
measuring one or more of turbidity, fluorescence, and colorimetric change of the incubated mixture to determine a result, wherein:
a change in the one or more of turbidity, fluorescence, and colorimetric change indicates a positive result, and
a positive result indicates the presence of the target nucleotide sequence.

12. The method of claim 11, wherein the reaction mixture further comprises:
a forward outer primer comprising a nucleic acid sequence selected from SEQ ID NOS. 1, 6, 11, 16, 21, 26, 31, 35, 40, 45, 50, 55, 60, and 65; and
a backward outer primer comprising a nucleic acid sequence selected from SEQ ID NOS. 2, 7, 12, 17, 22, 27, 32, 36, 41, 46, 51, 56, 61, and 66.

13. The method of claim 12, wherein the reaction mixture further comprises a Loop-F primer comprising a nucleic acid sequence selected from SEQ ID NOS. 5, 10, 15, 20, 25, 30, 39, 44, 49, 54, 59, 64, and 69.

14. The method of claim 13, wherein:
the forward inner primer comprises the nucleic acid sequence of SEQ ID NO. 3;
the backward inner primer comprises the nucleic acid sequence of SEQ ID NO. 4;
the forward outer primer comprises the nucleic acid sequence of SEQ ID NO. 1;
the backward outer primer comprises the nucleic acid sequence of SEQ ID NO. 2; and
the Loop-F primer comprises the nucleic acid sequence of SEQ ID NO. 5.

15. A nucleic acid screening kit to detect the presence of a target nucleotide sequence in a subject, the nucleic acid screening kit comprising:
a first reaction tube comprising:
a forward inner primer comprising a nucleic acid sequence selected from SEQ ID NOS. 3, 8, 13, 18, 23, 28, 33, 37, 42, 47, 52, 57, 62, and 67;
a backward inner primer comprising a nucleic acid sequence selected from SEQ ID NOS. 4, 9, 14, 19, 24, 29, 34, 38, 43, 48, 53, 58, 63, and 68;
a DNA polymerase with strand displacing activity;
dNTPs; and
a pH indicator;
a positive control tube comprising:
a forward inner primer comprising a nucleic acid sequence selected from SEQ ID NOS. 3, 8, 13, 18, 23, 28, 33, 37, 42, 47, 52, 57, 62, and 67;
a backward inner primer comprising a nucleic acid sequence selected from SEQ ID NOS. 4, 9, 14, 19, 24, 29, 34, 38, 43, 48, 53, 58, 63, and 68;
a DNA polymerase with strand displacing activity;
dNTPs;
a pH indicator; and
a synthetic nucleotide sequence substantially identical to the target nucleotide sequence; and
a negative control tube comprising:
a forward inner primer comprising a nucleic acid sequence selected from SEQ ID NOS. 3, 8, 13, 18, 23, 28, 33, 37, 42, 47, 52, 57, 62, and 67;
a backward inner primer comprising a nucleic acid sequence selected from SEQ ID NOS. 4, 9, 14, 19, 24, 29, 34, 38, 43, 48, 53, 58, 63, and 68;
a DNA polymerase with strand displacing activity;
dNTPs; and
a pH indicator.

16. The nucleic acid screening kit of claim 15, wherein each of the first reaction tube, the positive control tube, and the negative control tube further comprises a Loop-F primer comprising a nucleic acid sequence selected from SEQ ID NOS. 5, 10, 15, 20, 25, 30, 39, 44, 49, 54, 59, 64, and 69.

17. The nucleic acid screening kit of claim 15, further comprising a second reaction tube comprising:
a forward inner primer comprising a nucleic acid sequence selected from SEQ ID NOS. 3, 8, 13, 18, 23, 28, 33, 37, 42, 47, 52, 57, 62, and 67;
a backward inner primer comprising a nucleic acid sequence selected from SEQ ID NOS. 4, 9, 14, 19, 24, 29, 34, 38, 43, 48, 53, 58, 63, and 68;
a DNA polymerase with strand displacing activity;
dNTPs; and
a pH indicator.

18. The nucleic acid screening kit of claim 17, wherein each of the first reaction tube, the second reaction tube, the positive control tube, and the negative control tube further comprises a Loop-F primer comprising a nucleic acid sequence selected from SEQ ID NOS. 5, 10, 15, 20, 25, 30, 39, 44, 49, 54, 59, 64, and 69.

19. A nucleic acid screening kit to detect the presence of a target nucleotide sequence in a subject, the nucleic acid screening kit comprising:
a first reaction tube comprising:
a forward inner primer comprising a nucleic acid sequence selected from SEQ ID NOS. 3, 8, 13, 18, 23, 28, 33, 37, 42, 47, 52, 57, 62, and 67;
a backward inner primer comprising a nucleic acid sequence selected from SEQ ID NOS. 4, 9, 14, 19, 24, 29, 34, 38, 43, 48, 53, 58, 63, and 68;
a forward outer primer comprising a nucleic acid sequence selected from SEQ ID NOS. 1, 6, 11, 16, 21, 26, 31, 35, 40, 45, 50, 55, 60, and 65; and
a backward outer primer comprising a nucleic acid sequence selected from SEQ ID NOS. 2, 7, 12, 17, 22, 27, 32, 36, 41, 46, 51, 56, 61, and 66;
a DNA polymerase with strand displacing activity;
dNTPs; and
a pH indicator;
a positive control tube comprising:
a forward inner primer comprising a nucleic acid sequence selected from SEQ ID NOS. 3, 8, 13, 18, 23, 28, 33, 37, 42, 47, 52, 57, 62, and 67;
a backward inner primer comprising a nucleic acid sequence selected from SEQ ID NOS. 4, 9, 14, 19, 24, 29, 34, 38, 43, 48, 53, 58, 63, and 68;
a forward outer primer comprising a nucleic acid sequence selected from SEQ ID NOS. 1, 6, 11, 16, 21, 26, 31, 35, 40, 45, 50, 55, 60, and 65; and
a backward outer primer comprising a nucleic acid sequence selected from SEQ ID NOS. 2, 7, 12, 17, 22, 27, 32, 36, 41, 46, 51, 56, 61, and 66;
a DNA polymerase with strand displacing activity;
dNTPs;
a pH indicator; and
a synthetic nucleotide sequence substantially identical to the target nucleotide sequence; and
a negative control tube comprising:
a forward inner primer comprising a nucleic acid sequence selected from SEQ ID NOS. 3, 8, 13, 18, 23, 28, 33, 37, 42, 47, 52, 57, 62, and 67;
a backward inner primer comprising a nucleic acid sequence selected from SEQ ID NOS. 4, 9, 14, 19, 24, 29, 34, 38, 43, 48, 53, 58, 63, and 68;
a forward outer primer comprising a nucleic acid sequence selected from SEQ ID NOS. 1, 6, 11, 16, 21, 26, 31, 35, 40, 45, 50, 55, 60, and 65; and
a backward outer primer comprising a nucleic acid sequence selected from SEQ ID NOS. 2, 7, 12, 17, 22, 27, 32, 36, 41, 46, 51, 56, 61, and 66;
a DNA polymerase with strand displacing activity;
dNTPs; and
a pH indicator.

20. The nucleic acid screening kit of claim 19, further comprising a second reaction tube comprising:
a forward inner primer comprising a nucleic acid sequence selected from SEQ ID NOS. 3, 8, 13, 18, 23, 28, 33, 37, 42, 47, 52, 57, 62, and 67;
a backward inner primer comprising a nucleic acid sequence selected from SEQ ID NOS. 4, 9, 14, 19, 24, 29, 34, 38, 43, 48, 53, 58, 63, and 68;
a forward outer primer comprising a nucleic acid sequence selected from SEQ ID NOS. 1, 6, 11, 16, 21, 26, 31, 35, 40, 45, 50, 55, 60, and 65; and
a backward outer primer comprising a nucleic acid sequence selected from SEQ ID NOS. 2, 7, 12, 17, 22, 27, 32, 36, 41, 46, 51, 56, 61, and 66;
a DNA polymerase with strand displacing activity;
dNTPs; and
a pH indicator.
